(19) United States
(12) Patent Application Publication
(10) Pub. No.: US 2014/0275874 A1
(43) Pub. Date: Sep. 18, 2014

(54) SYSTEM AND METHOD FOR CHARGING A WIRELESS PULSE OXIMETER

(71) Applicant: Covidien LP, Mansfield, MA (US)

(72) Inventor: Charles Haisley, Boulder, CO (US)

(21) Appl. No.: 13/803,831

(22) Filed: Mar. 14, 2013

(51) Int. Cl. H02J 7/00; A61B 5/1455

(52) U.S. Cl. CPC H02J 7/0042; A61B 5/14551
USPC 600/323; 320/107; 600/309

(57) ABSTRACT

Methods and systems are provided for recharging a power module of a sensor (e.g., a wireless sensor). The system may include a charging station, which may receive and recharging the power module of the sensor. The charging station may be configured to recharge the power module directly or inductively. Furthermore, the charging station may be configured to recharge the power module while the power module is removed from the sensor or while the power module is operatively coupled to the sensor. Additionally, the charging station may be a component of a monitoring device, which may operate in combination with the sensor.

### BACKGROUND

The present disclosure relates generally to medical devices, and more particularly, to medical devices that monitor physiological parameters of a patient, such as pulse oximeters.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed or scattered. More specifically, the light passed through the tissue is typically selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

Wireless sensors have been developed for use in measuring physiological parameters of a patient. Powering of these devices may present a challenge as there are no wires connected to the sensor available to provide power to the sensors. While internal power sources such as batteries may be utilized, problems may exist in which the internal power source is drained, and the internal power source must be recharged or replaced to continue sensor operation.

### BRIEF DESCRIPTION OF THE DRAWINGS

Advantages of the disclosed techniques may become apparent upon reading the following detailed description and upon reference to the drawings in which:

FIG. 1 illustrates a perspective view of a patient monitoring system including a patient monitor and a wireless sensor, in accordance with an embodiment;

FIG. 2 illustrates a block diagram of the patient monitor and the wireless sensor of FIG. 1, in accordance with an embodiment;

FIG. 3 illustrates a perspective view of the patient monitor of FIG. 1 and a power module of the wireless sensor of FIG. 1, in accordance with an embodiment;

FIG. 4 illustrates a perspective view of the patient monitor of FIG. 1 and a power module of the wireless sensor of FIG. 1, in accordance with an embodiment;

FIG. 5 illustrates a perspective view of the patient monitor of FIG. 1 and a power module of the wireless sensor of FIG. 1, in accordance with an embodiment;

FIG. 6 illustrates a perspective view of the patient monitor of FIG. 1, a dongle configured to couple to the patient monitor, and a power module of the wireless sensor of FIG. 1, in accordance with an embodiment;

FIG. 7 illustrates a perspective view of a charging device, including an AC adapter, and a power module of the wireless sensor of FIG. 1, in accordance with an embodiment;

FIG. 8 illustrates a perspective view of a charging device, including an AC adapter, and a power module of the wireless sensor of FIG. 1, in accordance with an embodiment;

FIG. 9 illustrates a perspective view of a charging device, including an AC adapter, and a power module of the wireless sensor of FIG. 1, in accordance with an embodiment; and

FIG. 10 illustrates a perspective view of a cordless charger and a power module of the wireless sensor of FIG. 1, in accordance with an embodiment.

### DETAILED DESCRIPTION OF THE SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, wireless sensors may be used in conjunction with monitoring devices to monitor physiological parameters of a patient. Unfortunately, powering wireless sensors may present a challenge as there are no wires connected to the sensor available to provide power to the sensors. For example, an internal power source of the sensor, such as a battery, may drain with sensor use and may need to be recharged or replaced to continue sensor operation. In some situations, it may be desirable to recharge the power source, rather than replace it, to minimize cost and waste. However, recharging the power source may include removing the sensor from the patient and/or removing the power source from the sensor and recharging the power source at a central charging location. For example, a medical facility may have a designated area to recharge power sources. Unfortunately, recharging the power source at a central charging location may increase a chance of exposing the power source to contamination. As such, it may be desirable to recharge the power source at a charging station that is designated (e.g., dedicated) for the sensor and that is local to the patient, rather than at a central charging station.

Accordingly, the present disclosure is generally directed to techniques for recharging a power source of a wireless sensor at a charging station designated for the wireless sensor and local to the patient. As used herein, a charging station that is designated for a sensor is a charging device that is intended to be used with only that sensor for a period of time (e.g., while the sensor is in use with the same patient). For example, a sensor may include a power module having the power source, which may be removably attached to the sensor. In certain embodiments, the power module may be removed from the sensor and may be operatively coupled to a charging station for recharging. In other embodiments, the power module may be integral with the sensor (e.g., non-separable from the sensor). Generally, the charging station may include a receiving module for receiving the power module of the sensor (e.g., a plug-in connector, a snap-in inductive charging port, an inductive charging receptacle, etc.), a power source, a power transmission module to transmit power from the power source to the power module of the sensor, and processing circuitry (e.g., a processor) to monitor the recharging. In some embodiments, two or more power modules may be provided for each sensor, such that when one power module is removed for recharging, a second power module may be coupled to the sensor to continue sensor operation. In such embodiments, the designated charging station may be suitable to recharge any of the power modules for the given sensor.

In some embodiments, a monitoring device that operates in conjunction with the sensor may include the charging station. For example, a pulse oximetry monitor may be configured to recharge the power module of a pulse oximetry sensor. In certain embodiments, a processor of the monitoring device may be configured to monitor the recharging of the power module. To facilitate the power transmission between the monitoring device and the power module, the charging station of the monitoring device may include a plug-in connector, a snap-in inductive charging port, an inductive charging receptacle, or the like that is configured to receive and transmit charging signals to the power module. In other embodiments, the monitoring device may be configured to transmit power to the power module via a dongle (e.g., an adapter) interfacing between a sensor port of the monitoring device and the power module. The dongle is a device configured to plug into a port of the monitoring device and to bridge communication between the monitoring device and the sensor. In this manner, the dongle and the monitoring device in combination may function as the charging station. That is, the monitoring device may provide the power source and/or the processing circuitry for the charging station. The dongle may also be constructed to include a plug-in connector, a snap-in inductive charging port, and/or an inductive charging receptacle to facilitate the power transmission to the power module.

In certain embodiments, the charging station may be separate from the monitoring device. In such embodiments, the charging station may include a power adapter (e.g., an AC adapter), an energy harvesting power supply (e.g., a motion generated energy harvesting, thermoelectric generated energy harvesting, etc.), or any suitable power source. In these embodiments, the charging station may be a device external from the monitoring device but still local to the patient (e.g., in a room with the patient) and designated for the particular sensor.

In other embodiments, the power module may be recharged while physically coupled to the sensor. That is, the power module may be recharged without being removed from the sensor. In this manner, recharging may occur while the sensor is in use with the patient. This may be desirable to minimize cost and/or maximize the availability of the power modules. In particular, in some embodiments, a second power module may be provided to replace a power module that is removed from the sensor to be recharged. Accordingly, in embodiments in which the power module may be recharged while coupled to the sensor, a second power module may not be needed. In certain embodiments, a charging station may be provided that is configured to couple with the power module while the sensor is applied to the patient. In some embodiments, the external charging station, such as the power adapter, may include a cable having a length suitable to reach the patient. In other embodiments, an adapter may be provided that interfaces with the dongle and couples to the power module. In this manner, the adapter may enable the monitoring device to charge the power module while the power module is coupled to the sensor. Additionally, in one embodiment, a cordless charging station may be provided. For example, a cordless USB battery charger may couple with and transmit power to the power module. The cordless charging station may be a patient-wearable charging station. Regardless of the type of charging station provided, the charging station may be designated for the one or more power modules of the sensor.

With the foregoing in mind, FIG. 1 illustrates a perspective view of a patient monitoring system 10 is illustrated in accordance with an embodiment. The patient monitoring system 10 may include a monitor 12, which may be operatively coupled to a sensor 14, to monitor physiological parameters of a patient. Although the illustrated embodiment of the patient monitoring system 10 relates to photoplethysmography or pulse oximetry, the patient monitoring system 10 may be configured to obtain a variety of medical measurements with a suitable medical sensor. For example, the patient monitoring system 10 may additionally or alternatively be configured to perform regional oximetry, determine patient electroencephalography (e.g., a bispectral (BIS) index), or any other physiological parameter such as tissue water fraction or hematocrit.

The monitor 12 may be configured to display calculated parameters on a display 16. As illustrated in FIG. 1, the display 16 may be integrated into the monitor 12. However, the monitor 12 may be configured to provide data via a port to a display (not shown) that is not integrated with the monitor 12. The display 16 may be configured to display computed physiological data including, for example, an oxygen saturation percentage, a pulse rate, and/or a plethysmographic waveform 18. As is known in the art, the oxygen saturation percentage may be a functional arterial hemoglobin oxygen saturation measurement in units of percentage SpO₂, while the pulse rate may indicate a patient's pulse rate in beats per minute. The monitor 12 may also display information related to alarms, monitor settings, and/or signal quality via indicator lights 20. Additionally, the monitor 12 may include a speaker 22 to provide audible information to a user relating to the patient monitoring system 10 (e.g., alarms).

To facilitate user input, the monitor 12 may include a plurality of control inputs 24. The control inputs 24 may include fixed function keys, programmable function keys, and soft keys. Specifically, the control inputs 24 may correspond to soft key icons in the display 16. Pressing control inputs 24 associated with, or adjacent to, an icon in the display may select a corresponding option. The monitor 12 may also include a casing 26. The casing 26 may aid in the protection of the internal elements of the monitor 12 from damage.

As noted above, the monitor 12 may be operatively coupled to the sensor 14. Accordingly, the monitor 12 may include a transceiver 28, which may enable for wireless signals to be transmitted to and received from the sensor 14. In this manner, the monitor 12 and the sensor 14 may communicate wirelessly. In certain embodiments, the monitor 12 may also include a sensor port 30, which may be configured to couple to a cable of the sensor 14 via a plug (not shown). That is, in some embodiments, the monitor 12 may be wirelessly coupled to the sensor 14 and/or may be physically coupled to another sensor (not shown) via the sensor port 30.

The sensor 14 may include one or more emitters 32 and one or more detectors 34, which will be described in more detail below with respect to FIG. 2, to acquire a physiological signal of a patient that can be used by the monitor 12 to calculate certain physiological characteristics of the patient. For example, the physiological signal may correspond to physiological characteristics such as the blood-oxygen saturation of hemoglobin in arterial blood, the volume of individual blood pulsations supplying the tissue, and/or the rate of blood pulsations corresponding to each heartbeat of a patient. The sensor may also include a sensor body 36 to house components of the sensor 14 (e.g., the emitter 32 and the detector 34). The sensor body 36 may be formed from any suitable material, including rigid and/or conformable materials, such as fabric, paper, rubber, or elastomeric compositions (including acrylic elastomers, polyimide, silicone rubber, celluloid, PMDS elastomer, polyurethane, polypropylene, acrylics, nitrile, PVC films, acetates, and latex).

Additionally, as will be discussed in greater detail below with respect to FIGS. 2-10, the sensor 14 may include a power module 38, for providing and/or monitoring power for use by the sensor 14. However, as will be described in more detail below, in some embodiments, the power module 38 may also include components of the sensor 14 to facilitate the acquisition of and/or the transmission of a physiological signal generated by the sensor 14. In certain embodiments, the power module 38 may be removably attached to the sensor body 36. For example, the power module 38 may be removably attached to an external surface of the sensor body 36 such that the power module 38 may be removed by sliding the power module 38 away from the sensor body 36, pulling the power module 38 away from the sensor body 36 via a tab or projection on the power module 38, pushing a button on the sensor body 36 and/or the power module 38 to release the power module, or similar actions. In other embodiments, the power module 38 may be disposed inside the sensor body 36 such that at least a portion of the sensor body 36 may be configured to move (e.g., be removed or slide open) to enable the power module 38 to be removed. In one embodiment, the power module 38 may be integral with the sensor 14 (e.g., non-separable). Additionally, in some embodiments, the power module 38 may be disposed externally from the sensor body 36 and attached to the sensor 14 via a lead (FIGS. 8-10).

In certain embodiments, as will be discussed in more detail below, the monitor 12 may include a charging station 40 to receive and charge the power module 38 of the sensor 14. By way of example, the charging station 40 may include a plug-in connector, a snap-in connector, a receptacle, or a similar, and may be configured to transmit charging signals to the power module 38 directly and/or inductively. Furthermore, in some embodiments, as will be described in more detail below, the sensor port 30 in combination with a dongle (FIG. 6) may function as the charging station 40 to charge the power module 38.

Turning to FIG. 2, a block diagram of the patient monitoring system 10 is illustrated in accordance with an embodiment. Specifically, certain components of the sensor 14 and the monitor 12 are illustrated in FIG. 2. As noted above, the sensor 14 may include the emitter 32 and the detector 34. The emitter 32 may emit light into a patient 50, which may be reflected by or transmitted through the patient 50 and subsequently detected by the detector 34. In some embodiments, the emitter 32 may emit one or more different wavelengths of light. For example, the emitter 32 may emit red wavelengths between approximately 600 nanometers (nm) and 700 nm and/or infrared wavelengths between approximately 800 nm and 1000 nm. In other embodiments, the emitter 32 may emit a red wavelength between approximately 620 nm and 700 nm (e.g., 660 nm), a far red wavelength between approximately 690 nm and 770 nm (e.g., 730 nm), and an infrared wavelength between approximately 860 nm and 940 nm (e.g., 900 nm). Other wavelengths may include, for example, wavelengths between approximately 500 nm and 600 nm and/or 1000 nm and 1100 nm. Alternative light sources may be used in other embodiments. For example, a single wide-spectrum light source may be used, and the detector 34 may be configured to detect certain wavelengths of light. In another example, the detector 34 may detect a wide spectrum of wavelengths of light, and the monitor 12 may process only those wavelengths which are of interest for use in measuring, for example, water fractions, hematocrit, or other physiologic parameters of the patient 50. It should be understood that, as used herein, the term “light” may refer to one or more of ultrasound, radio, microwave, millimeter wave, infrared, visible, ultraviolet, gamma ray, or X-ray electromagnetic radiation, and may also include any wavelength within the radio, microwave, millimeter wave, infrared, visible, ultraviolet, or X-ray spectra, and that any suitable wavelength of light may be appropriate for use with the present disclosure.

Additionally, the sensor 14 may include an encoder 52, which may contain information about the sensor 14. For example, the encoder 52 may contain information regarding what type of sensor it is (e.g., whether the sensor is intended for placement on a forehead or digit) and the wavelengths of light emitted by the emitter 32. This information may enable the monitor 12 to select appropriate algorithms and/or calibration coefficients for calculating the physiological characteristics of the patient 50. Additionally, the encoder 52 may include information relating to proper charging of the sensor 14. For example, the encoder 52 may include information relating to the number and/or type of power sources 54 (e.g., rechargeable batteries) of the power module 38. In some embodiments, the encoder 52 may also store information relating to a minimum charge threshold of the power source 54. Additionally, the encoder 52 may be programmed with an identification number for the power module 38 and/or the sensor 14. Further, in some embodiments, the encoder 52 may be programmed with an identification number of a designated charging station (e.g., charging device) for the sensor 14. The encoder 52 may, for instance, be a memory, a coded resistor, EEPROM, or other coding devices that may provide a signal to the monitor 12 relating to the characteristics of the sensor 14. The monitor 12 may include a reader/decoder 56 that may read and/or decode information from the encoder 52 to provide the monitor 12 with information about the sensor 14.

Signals from the detector 34 and the encoder 52 (if utilized) may be transmitted to the monitor 12 via a transmitter 64 that may be located in a transceiver 66. The transceiver 66 may also include a receiver 68 that may be used to receive signals from the monitor 12. As noted above, the monitor 12 may include the transceiver 28. The transceiver 28 may include a receiver 70 to receive transmitted signals from the transmitter 64 of the sensor 14 and a transmitter 72 to transmit signals to the receiver 68 of the sensor 14. In this manner, the sensor 14 may wirelessly communicate with the monitor 12. The monitor 12 may also include one or more processors 74 coupled to an internal bus 76. Also connected to the bus 76 may be a random-access memory (RAM) 78, the display 16, the speaker 22, and the control inputs 24. The monitor 12 may also include a time processing unit (TPU) 80 that may provide timing control signals to a light drive (e.g., light drive circuitry) 82, which may control (e.g., via the transmitter 28) when the emitter 32 is activated, and if multiple light sources are used, the multiplexed timing for the different light sources. The TPU 80 may also control the gating-in of signals from the detector 34 through an amplifier 84 and a switching circuit 86. These signals may be sampled at the proper time, depending upon which light source is illuminated. The received signal from the detector 52 may be passed through an amplifier 88, a low pass filter 90, and an analog-to-digital converter (A/D) 92 for amplifying, filtering, and digitizing the signals from the sensor 14. The digital data may then be stored in a queued serial module (QSM) 94 for later downloading to the RAM 78 as the QSM 94 fills up. In an embodiment, there may be multiple parallel paths of separate amplifier, filter, and A/D converters for multiple wavelengths or spectra received.

In an embodiment, based at least in part upon the received signals corresponding to the light received by the detector 34, the processor 74 may calculate physiological characteristics of the patient 50, such as the oxygen saturation of the patient 50, using various algorithms. These algorithms may use coefficients, which may be empirically determined, and may correspond to the wavelengths of light used. The algorithms may be stored in a read-only memory (ROM) 96 and accessed and operated according to processor 74 instructions. As noted above, the monitor 12 may also receive information from the encoder 52, which may be decoded by the decoder 56 and provided to the processor 74. In particular, the decoded signals may provide information to the processor 74 such as the sensor type and the wavelengths of light emitted by the emitter 32, so that proper calibration coefficients and/or algorithms to be used for calculating physiological characteristics of the patient 50 may be selected and utilized by the processor 74.

The monitor 12 may also include a power source 100 that may be used to transmit power to the components of the monitor 12. In certain embodiments, the power source 100 may be one or more batteries, such as a rechargeable battery. The battery may be user-removable or may be secured within the casing 26 of the monitor 12. Use of a battery may, for example, enable the monitor 12 to be highly portable, thus allowing a user to carry and use the monitor 12 in a variety of situations and locations. In addition to or instead of the power source 100, the monitor 12 may include a power adapter 102 that may enable the monitor 12 to be operatively coupled to an external power source 104 (e.g., AC power from an electrical outlet). The power adapter 102, in conjunction with the external power source 104, may directly power the monitor 12 and/or may recharge the power source 100, if utilized.

As noted above, in certain embodiments, the monitor 12 may include the charging station 40 that may be configured to receive and recharge the power module 38 of the sensor 14. In certain embodiments, the charging station 40 may include processing circuitry, a processor, and/or a transceiver for monitoring and/or communicating with the power module 38. The charging station 40 may be coupled to the power source 100 and/or the power adapter 102 to facilitate the transmission of power from the monitor 12 to the power module 38. For example, the charging station 40 may include a power transmission module 106 that may be configured to transmit power wirelessly (e.g., inductively) and/or directly (e.g., via one or more electrical contacts). In particular, the power transmission module 106 may receive power from the power source 100 and/or the external power source 104 (e.g., via the power adapter 102) and may transmit power wirelessly and/or directly to a power receiving module 108 of the power module 38 of the sensor 14. In certain embodiments, the power transmission module 106 may also include circuitry to process the received power such that it is suitable to be received by the power receiving module 108 and/or to be used by the sensor 14. Additionally, as will be discussed in more detail below, the power transmission module 106 and the power receiving module 108 may include one or more inductors, one or more electrical contacts, and/or any other suitable components to facilitate the power transmission between the power transmission module 106 and the power receiving module 108. That is, in certain embodiments, the power transmission module 106 and/or the power receiving module 108 may include components suitable for wireless power transmission and components suitable for wired power transmission.

In some embodiments, in addition to the power source 54 and the power receiving module 108, the power module 38 of the sensor 14 may also include other components of the sensor 14 to facilitate the acquisition of and/or the transmission of the physiological signals. For example, the power module 38 may also include the transceiver 66, the encoder 52, and/or light drive circuitry (not shown). These may be desirable in certain embodiments, such as, for example, embodiments of the sensor 14 that may not be configured to operate wirelessly and/or may not include components to enable calibration of the sensor 14. Thus, providing the power module 38 may enable the sensor 14 to operate wirelessly (e.g., via the transceiver 66 and/or the light drive circuitry) and may enable calibration of the sensor 14 (e.g., via the encoder 52). This embodiment of the power module 38 may be also desirable for use with disposable sensors 14, which may not include more costly components such as, for example, the transceiver 66, the encoder 52, and/or the light drive circuitry. In particular, the power module 38, which may be reusable, may be reused with various disposable sensors 14, which may reduce manufacturing costs.

Additionally, the power module 38 may include a charging control circuit 110. The charging control circuit 110 may, for example, enable the adaptive control of energy received by the power receiving module 108 for use in the power source 54 of the sensor 14. The power source 54 may be used to transmit power to components located in the sensor 14. In one embodiment, the power source 54 may be one or more batteries, such as a rechargeable battery. The battery may be, for example, a lithium ion, lithium polymer, nickel-metal hydride, or nickel-cadmium battery. Alternatively, the power source 54 may be one or more capacitors configured to store charge. The charging control circuit 110 may, for example, include a processing circuit and/or a processor that may determine the current level of charge remaining in the power source 54, as well as the amount and/or rate of power received by the power receiving module 108 (e.g., from the power transmission module 106). For example, the charging control circuit 110 may compare the determined level of charge of the power source 54 to a threshold and may determine that the power source 54 is low on power if the level of charge is below the threshold. By way of example, the charging control circuit 110 may determine that the power source 54 is low on power if between approximately zero percent and fifty percent, five percent and forty percent, or ten percent and thirty percent of the total charge of the power source 54 is remaining.

In response to determining that the power source 54 is low on power, the charging control circuit 110 may be configured to generate a user-perceivable indication. Providing a user-perceivable indication may be desirable to alert a user that the power source 54 may need charge and, thus, may prompt the user to provide the power module 54 to a charging device (e.g., the charging station 40). In certain embodiments, the charging control circuit 110 may cause a speaker 114 of the sensor 14 to emit an audible indication and/or may cause one or more indicator lights 116 (e.g., LEDs) of the sensor 14 to emit light in response to determining that the power source 54 is low on power. Additionally or alternatively, the charging control circuit 110 may cause a display 118 of the sensor 14 to display a user-perceivable indication (e.g., an image, a symbol, a textual message, or the like) relating to the power level of the power source 54. In one embodiment, the display 118 may be an electronic ink (E-ink) display. In some embodiments, the display 118 may display a battery indicator, which may be at least periodically updated by the charging control circuit 110 in response to a detected change in the power level of the power source 54. In one embodiment, the charging control circuit 110 may be configured to update the battery indicator continuously. In other embodiments, the charging control circuit 110 may be configured to update the battery indicator approximately every five seconds to ten minutes, ten seconds to five minutes, fifteen seconds to three minutes, twenty seconds to two minutes, or thirty seconds to one minute. Additionally, the charging control circuit 110 may be configured to alter the battery indicator when the charging control circuit 110 determines that the power source 54 is low on power. For example, the charging control circuit 110 may cause the battery indicator to change colors, increase in size, and/or flash.

Additionally, the charging control circuit 110 may determine when the power source 54 is fully charged and may provide a suitable user-perceivable indication of the completion of charge (e.g., via the speaker 114, the indicator lights 116, and/or the display 118). In some embodiments, the user-perceivable indications of the completion of charge may be different from the user-perceivable indications of low power. By way of example, the battery indicator of the display 118 may be displayed as full (e.g., fully shaded or showing all battery bars) and/or one of the one or more indicator lights 116 may emit light of a different color than another of the one or more indicator lights 116 that is emitted when the power source 54 is low on power. Additionally or alternatively, the charging control circuit 110 may be configured to generate and/or transmit a signal relating to the power level of the power source 54 to a charging device (e.g., the monitor 12). That is, in some embodiments, the charging control circuit 110 may include a transmitter to facilitate the transmission of wireless signals to the charging station 40 and/or to the receiver 70 of the monitor 12. Alternatively, the charging control circuit 110 may cause the transmitter 64 of the sensor 14 to transmit a wireless signal. In response to receiving the signal relating to the power source 54, the display 16 of the monitor 12 may display an indication relating to the power level of the power source 54, which may be, for example, a battery indicator. Additionally, the monitor 12 may be configured to emit one or more of the indicator lights 20 when the power source 54 is determined to be fully charged and/or display any other suitable indication (e.g., image, symbol, or textual message).

In certain embodiments, the charging control circuit 110 may also be configured to generate and/or transmit an identification signal identifying the power module 38 to the charging station 40. It may be desirable to provide the identification signal to verify whether the power module 38 is provided to the designated charging station 40. That is, while the charging station 40 may be designated for the sensor 14, a power module of a different sensor may inadvertently be provided to the charging station 40 and/or the power module 38 of the sensor 14 may be inadvertently provided to a different charging device, which may increase a risk of exposing the power module 38 to contamination. Additionally, the identification signal, if verified, may activate the charging station 40. That is, until the charging station 40 receives the identification signals and verifies that the power module 38 is a device suitable to be charged by the charging station 40, the charging station 40 may remain in an “off” state. For example, in embodiments in which the charging station 40 is configured to inductively charge the power module 40, the power transmission module 106 may not transmit wireless electromagnetic charging signals while in the “off” state. In addition, in embodiments in which the charging station 40 is configured to transmit power directly (e.g., via electrical contacts) to the power module 38, the power transmission module 106 may not transmit charging signals. In certain embodiments, the charging control circuit 110 may be configured to generate and/or transmit the identification signal to the charging station 40 when the power module 38 is physically coupled to the charging station 40 (e.g., via a snap-in connector), or when the power module 38 is within range of a wireless electromagnetic charging signal from the charging station 40. By way of example, the range of the wireless electromagnetic charging signals may be between approximately zero centimeters and three meters, five centimeters and two meters, ten centimeters and one meter, or fifteen centimeters to fifty centimeters, or any other range.

In some embodiments, the identification signal may include the identification number corresponding to the power module 38 and/or the sensor 14 that is stored in the encoder 52. For example, the charging control circuit 110 may read the identification number from the encoder 52 and may transmit the identification number to the receiver 70 of the monitor 12. A memory of the monitor 12 (e.g., the RAM 78 and/or the ROM 96) may be programmed with the identification number or identification numbers (if more than one power module 38 is used for a given sensor 14) that may be used with the charging station 40 or the monitor 12. In other embodiments, the power module 38 and/or the charging station 40 may be programmed with a radio-frequency identification (RFID) label, and the identification signal may be a RFID signal. That is, in certain embodiments, the charging station 40 may be configured to receive and analyze the identification signal independent of the monitor 12. Thus, based at least in part upon the identification number and/or the RFID label, the charging control circuit 110, the charging station 40, and/or the processor 74 of the monitor 12 may determine whether the power module 38 is provided to the designated charging station 40.

In response to a determination that the power module 38 is not provided to the designated charging station 40, the power module 38 and/or the charging station 40 may provide a user-perceivable indication that the charging station 40 is not intended to be used with the power module 38. Providing an indication of a mismatched power module 38 and charging station 40 may alert a user that it may be desirable to disinfect the power module 38 before reattaching the power module 38 to the sensor 14. For example, one of the one or more indicator lights 116 may emit light in a color corresponding to a mismatch (e.g., red or any other suitable color). Additionally, the speaker 114 of the power module 38 may be configured to emit an audible indication, such as a beep. Further, in some embodiments, the display 16 of the monitor 12 and/or the display 118 (e.g., an E-ink display) of the power module 38 may be configured to provide an error indication or an error message. Alternatively, in response to determining that the power module 38 is provided to the designated charging station 40, the power module 38 and/or the charging station 40 may provide an indication of a matched power module 38 and charging station 40. For example, one of the one or more indicator lights 116 of the power module 40 may emit light in a color corresponding to a match (e.g., green or any other suitable color). Additionally, in certain embodiments, the determination that the power module 38 is provided to the designated charting station 40 may activate the power transmission module 106 to initiate the power transmission.

In addition to determining whether the power module 38 is provided to a designated charging device, the charging control circuit 110 may also be configured to determine whether the power receiving module 108 is failing to be charged by the charging station 40. In particular, the charging control circuit 110 may determine when the power receiving module 108 should be receiving power (e.g., for charging) from the charging station 40 (e.g., from the power transmission module 106). For example, the charging control circuit 110 may determine that the power receiving module 108 should be receiving power (e.g., for charging) when the power module 38 is coupled to the charging station 40 (e.g., via a snap-in connector). Additionally or alternatively, the charging control circuit 110 may determine that the power receiving module 108 should be receiving charging power when the power module 38 is within range of an inductive charging signal from the charging station 40. As noted above, the range of the inductive charging signals may be between approximately zero centimeters and three meters, five centimeters and two meters, ten centimeters and one meter, or fifteen centimeters to fifty centimeters, or any other range. In response to determining that the power receiving module 108 is failing to be charged by the charging station 40, the charging control circuit 110 may generate a user-perceivable error indication (e.g., symbol or textual message on the display 118, an audible alarm via the speaker 114, and/or a visual indication via the one or more indicator lights 116). Additionally or alternatively, the charging control circuit 110 may generate an error signal, which may be transmitted to the charging station 40 and/or the processor 74 of the monitor 12 for the generation of a corresponding error indication by the charging station 40 or the monitor 12, respectively. The error indication may indicate to a user that the power module 38 and/or the charging station 40 is potentially malfunctioning, and may direct the user, for example, to replace the power module 38 and/or to utilize a different charging station 40.

As noted above, the power module 38 may be removed from the sensor 14 and may be coupled to the charging station 40 to facilitate the power transmission from the power transmission module 106 to the power receiving module 108. Accordingly, the charging station 40 may include a receiving module 120 configured to couple to (e.g., receive) the power module 38. For example, as will be described in more detail below with respect to FIGS. 3-5, the receiving module 120 may include a plug-in connector, an inductive charging port, an inductive charging receptacle, and/or any other suitable connection. Accordingly, the power module 38 may include a housing that may be removably coupled to the sensor 14 and that may include a plug configured to be inserted into a connector of the charging station 40. Additionally or alternatively, the housing of the power module 38 may have a geometry that is configured to be inserted into an inductive charging port and/or an inductive charging receptacle of the charging station 40.

For example, FIG. 3 illustrates an embodiment of the power module 38 that is configured to plug into a connector of the charging station 40. As illustrated, the power module 38 includes a housing 130 and is removed from the sensor 14. The housing 130 may be constructed from any suitable material, including rigid and/or conformable materials, such as rubber or elastomeric compositions (including acrylic elastomers, polyimide, silicone rubber, celluloid, PMDS elastomer, polyurethane, polypropylene, acrylics, nitrile, PVC films, acetates, and latex). As illustrated, the housing 130 may include a tab 132 (e.g., a projection) to facilitate the removal of the housing 130 from the sensor body 36 of the sensor 14. The housing 130 may also include the one or more indicator lights 116 (e.g., LEDs) and the display 118, as described above. As illustrated, the display 118 includes a battery indicator 134. The battery indicator 134 may include a series of lines, as shown in FIG. 3, and/or a shaded area corresponding to an approximate level of charge remaining in the power source 54. In some embodiments, the display 118 may be configured to display a percentage of the total charge of the power source 54 remaining and/or an approximate time remaining before the power source 54 is low on power (e.g., below a predetermined threshold or no charge remaining). Additionally, the power module 38, as illustrated, includes three indicator lights 116 (e.g., LEDs), which may be configured to emit different wavelengths (e.g., different colors). For example, a first indicator light 136 may emit a first color (e.g., green) when the power source 54 is fully charged, a second indicator light 138 may emit a second color (e.g., yellow) when the power source 54 is receiving charge, and a third indicator light 140 may emit a third color (e.g., red) when the power source 54 is low on charge (e.g., below a predetermined threshold for low charge). In certain embodiments, a single indicator light 116 may be configured to emit multiple colors. Additionally, the charging control circuit 110 may cause, for example, the third indicator light 140 to flash in response to determining that the power source 54 is failing to be charged and/or that the power source 54 is not provided to the designated charging station 40.

In certain embodiments, the housing 130 of the power module 38 may also include a connector 142 (e.g., a power plug or “male” connector). In some embodiments, the connector 142 may be configured to fold and/or swivel about the housing 130 to minimize the size and/or bulkiness of the housing 130. This may be desirable in certain embodiments to facilitate the attachment of the power module 38 to the sensor 14. Additionally, in certain embodiments, the connector 142 may couple to the circuitry of the sensor 14 to provide power to one or more components of the sensor 14 (e.g., the emitter 32 and the detector 34). In other embodiments, the housing 130 may include one or more additional electrical contacts (not shown) to couple to the circuitry of the sensor 14. The connector 142 may be any suitable connector, such as a universal serial bus (USB) plug, a DC power plug, a coaxial DC power plug, a locking DC power plug, an AC power plug, or the like. The charging station 40 of the monitor 12 may include a mating connector 146 (e.g., a socket, receptacle, or “female” connector) configured to receive the connector 142 of the power module 38. Accordingly, the mating connector 146 may be a USB receptacle, a DC power receptacle, a coaxial DC power receptacle, a locking DC power receptacle, an AC power receptacle, or the like. However, it should be noted that the present embodiments also anticipate designing the connector 142 of the power module 38 as a receptacle (e.g., female connector) and the mating connector 146 of the charging station 40 as a plug (e.g., male connector).

As noted above, two or more power modules 38 may be provided for the sensor 14 to facilitate the charging of one power module 38, while another power module 38 is in use with the sensor 14. In some embodiments, three or more power modules 38 may be provided for the sensor 14 such that two power modules 38 may be charged simultaneously while another power module 38 is in use with the sensor 14. Accordingly, in certain embodiments, the charging station 40 may include two or more mating connectors 146. In the illustrated embodiment, the charging station 40 includes a first mating connector 148 and a second mating connector 150. As illustrated, the first and second mating connectors 148 and 150 are the same type of connector. However, in other embodiments, the first mating connector 148 may be one type of connector (e.g., a USB receptacle), and the second mating connector 150 may be another type of connector (e.g., a DC power receptacle). Additionally, the charging station 40 may include one or more indicator lights 152. In particular, the charging station 40, as illustrated, includes a first indicator light 154 proximate to the first mating connector 148 and a second indicator light 156 proximate to the second connector 150. Similar to the one or more indicator lights 116 of the sensor 14, the one or more indicator lights 152 of the charging station 40 may be configured to emit one or more colors in response to determining that the power source 54 is receiving charge, the power source 54 is fully charged, the power source 54 is failing to be charged, and/or the power source 54 is not provided to the designated charging station 40. In one embodiment, a respective indicator light 152 may flash while the power source 54 is receiving charge and may continuously emit light when the power source 54 is fully charged, or vice versa.

As noted above, the power module 38 may be configured to receive wireless electromagnetic charging signals in addition to, or instead of, wired charging signals. For example, FIG. 4 illustrates an embodiment of the power module 38 that is configured to snap into an inductive charging port 170 of the charging station 40. The inductive charging port 170 may include at least one primary inductor 172 (e.g., a coiled conductor or a coiled wire) that may receive power from the power source 100, the power adapter 102, or any other suitable power source. The primary inductor 172, when coupled to a power source, may create an electromagnetic field that may induce an electrical current in at least one secondary inductor 174 of the power module 38. This current may be utilized to recharge the power source 54 of the power module 38. In this manner, the charging station 40 may wirelessly recharge the power module 38.

To facilitate the positioning of the power module 38 into the inductive charging port 170, the housing 130 of the power module 38 and the inductive charging port 170 may be shaped with complementary geometries. That is, the housing 130 of the power module 38 and the inductive charging port 170 may be shaped in any suitable means to enable the power module 38 to be inserted into (e.g., snap into) the inductive charging port 170. In some embodiments, the housing 130 may also include the tab 132 and/or the connector 142, as described above with respect to FIG. 3. The geometry of the housing 130 may be designed to position the secondary inductor 174 in proximity of and in alignment with the primary inductor 172 to maximize the efficiency of the energy transfer. For example, the housing 130 may include a protrusion 176 having the secondary inductor 174 that may be configured to abut an indentation 178 (e.g., a cavity, a recess, a groove, etc.) having the primary inductor 172. Additionally, in certain embodiments, the housing 130 and/or the inductive charging port 170 may include one or more magnets (not shown) to facilitate the alignment of the primary and secondary inductors 172 and 174.

Similar to the mating connectors 146 as described above, the charging station 40 may also include two or more inductive charging ports 170. In particular, the charging station 40 may include a first inductive charging port 180 and a second inductive charging port 182. However, in other embodiments, the charging station 40 may include a combination of mating connectors 146 and inductive charging ports 170. For example, the charging station 40 may include the first mating connector 148 and the first inductive charging port 180. Additionally, the charging station 40 may include the one or more indicator lights 152 (e.g., the first and the second indicator lights 154 and 156).

In other embodiments, in addition to or instead of providing the mating connectors 146 and/or the inductive charging ports 170, the charging station 40 may include an inductive charging receptacle 200, as illustrated in FIG. 5. The inductive charging receptacle 200 may be desirable in certain embodiments because it may contain and charge one or more power modules 38, which may not include the connector 142 and/or a particular geometry of the housing 130 to snap into the inductive charging port 170. As illustrated, the inductive charging receptacle 200 may protrude from the casing 26 of the monitor 12. However, in other embodiments, the inductive charging receptacle 200 may be a cut-out portion of the casing 26. The inductive charging receptacle 200 may be a box, a bowl, or any other suitable vessel for holding one or more power modules 38. For example, the inductive charging receptacle 200 may be constructed to hold one, two, three, four, or any other suitable number of power modules 38. The primary inductor 172 may be disposed in any suitable location of the inductive charging receptacle 200. In one embodiment, the primary inductor 172 may be disposed in a wall of the inductive charging receptacle 200, such as the bottom wall 202.

The charging station 40 may also include the one or more indicator lights 152 (e.g., the first and the second indicator lights 154 and 156). However, as more than one power module 38 may be placed in the inductive charging receptacle 200, rather than in a connector (e.g., the mating connector 146 or the inductive charging port 170) having one of the indicator lights 152 in close proximity (e.g., adjacent to), a user may experience difficulty in determining which indicator light 152 corresponds to which power module 38. Accordingly, in certain embodiments, the charging station 40 and/or the processor 74 of the monitor 12 may determine the availability of the one or more indicator lights 152 and may send a signal to the charging control circuit 110 of the power module 38 relating to the appropriate indicator light 152 (e.g., the first indicator light 154 or the second indicator light 156) designated for the power module 38. For example, if the inductive charging receptacle 200 is empty and no indicator lights 152 are in use with a power module 38, the first power module 38 placed into the inductive charging receptacle 200 may be linked to the first indicator light 154. In some embodiments, to provide an indication to a user regarding the designated indicator light 152, the charging control circuit 110 may cause the display 118 to display an indication (e.g., a number, a symbol, an image, a textual message, etc.) relating to the designated indicator light 152, which may be based upon a signal received from the charging station 40 and/or the processor 74 of the monitor 12. Alternatively, each power module 38 may be configured to cause a particular indicator light 152 to emit light, rather than linking a power module 38 to an indicator light 152 each time a power module 38 is placed in the inductive charging receptacle 200.

While the embodiments described above with respect to FIGS. 3-5 relate to embodiments of the charging station 40 that are built into the monitor 12, other embodiments may provide a retrofit dongle that may couple to the monitor 12 to provide the charging station 40. For example, a retrofit dongle may couple to a connector of the monitor 12, such as the sensor port 30. This may be desirable in certain embodiments to reduce cost as the charging station 40 may be provided to existing monitors 12, rather than manufacturing a monitor 12 having the charging station 40. For example, FIG. 6 illustrates an embodiment of a dongle 220 including the charging station 40 that may be configured to couple to the sensor port 30 of the monitor 12. As illustrated, the dongle 220 includes a connector portion 222 that is configured to couple to the sensor port 30. However, it should be noted that in other embodiments the dongle 220 may be configured to couple to another connector or port of the monitor 12, such as a USB port or a serial port.

As noted above, the dongle 220 in combination with the monitor 12 may include the charging station 40. That is, the monitor 12 may provide power (e.g., via the power source 100 and/or the power adapter 102) to the dongle 220, and the dongle 220 may include the receiving module 120 to receive the power module 38 (e.g., the mating connector 146, the inductive charging port 170, and/or the inductive charging receptacle 200), the power transmission module 106 (e.g., the primary inductor 172), and the one or more indicator lights 152. In certain embodiments, the processor 74 of the monitor 12 may monitor and/or control the recharging. For example, the processor 74 may determine when the power module 38 is being charged and/or when the power module 38 is fully charged and may cause one of the indicator lights 152 to emit light in response to the determination of charging and/or fully charged. Additionally or alternatively, the dongle 220 may include processing circuitry and/or a processor to monitor the recharging. In certain embodiments, the dongle 220 may also include a memory (e.g., a tangible, non-transitory memory), which may store instructions or code for communicating with the monitor 12 and/or the sensor 14 and for recharging the power module 38. The processor of the dongle 220 and/or the processor 74 of the monitor 12 may be configured to read and implement the code stored in the memory of the dongle 220. Additionally, in certain embodiments, the dongle 220 may be configured to store data in the memory such as information relating to the recharging of the power module 38. For example, the dongle 220 may store the number of times each power module 38 has been recharged, as well as the corresponding identification number for each power module 38. Additionally, the dongle 220 may be configured to store information relating to any events in which the power module 38 failed to receive charging power.

As illustrated, the receiving module 120 of the dongle 220 includes the mating connector 146, as described above with respect to FIG. 3, that is configured to couple to the connector 142 of the power module 38. However, it should be noted that the receiving module 120 may additionally or alternatively include the inductive charging port 170, and/or the inductive charging receptacle 200, as described above with respect to FIGS. 4 and 5, respectively. That is, the dongle 220 may include the mating connector 146, the inductive charging port 170, and the inductive charging receptacle 200 in any suitable combination. Additionally, the dongle 220 may include any suitable number of each of the mating connector 146, the inductive charging port 170, and/or the inductive charging receptacle 200.

In addition to recharging the power module 38, the dongle 220 may be configured to wirelessly communicate with the sensor 14. For example, the dongle 220 may include a transceiver 228 to wirelessly communicate with the transceiver 66 of the sensor 14 using any suitable wireless standard. In particular, the transceiver 228 of the dongle 220 may wirelessly receive physiological signals from the sensor 14. The monitor 12 may receive the physiological signals from the dongle 220 and may calculate physiological parameters of the patient based at least in part upon the received physiological signals. Additionally, the monitor 12 may be configured to transmit emitter driving signals (e.g., to cause the emitter 32 of the sensor 14 to emit light) via the transceiver 228 of the dongle 220. Further, in certain embodiments, the transceiver 228 of the dongle 220 may be configured to receive signals from the encoder 52 of the sensor 14 and may transmit the signals to the decoder 56 and/or the processor 74 of the monitor 12. In this manner, the monitor 12 may wirelessly communicate with the sensor 14, calibrate the sensor 14, and/or control the operation of the sensor 14 via the dongle 220. This may be desirable, for example, in embodiments in which the monitor 12 does not include the transceiver 28.

As noted above, the charging station 40 may also be separate from the monitor 12. That is, the charging station 40 may include a power source and/or may receive power from a power source separate from the monitor 12. This may be desirable in certain embodiments, for example, in which the monitor 12 is not present (e.g., the sensor 14 may calculate the physiological parameters) or the monitor 12 is not equipped to receive and recharge the power module 38. For example, FIG. 7 illustrates an embodiment of the charging station 40 that may be configured to receive power from a power source 240. As illustrated, the power source 240 may be an AC power source 242 (e.g., an AC power socket). However, it should be noted that any suitable power source many be utilized. For example, the charging station 40 may include a battery and/or an energy harvesting power supply (e.g., a motion generated energy harvesting device, thermoelectric generated energy harvesting device, or the like). The charging station 40 may include an AC adapter 244, which may include a plug 246 configured to plug into the AC power source 242. In certain embodiments, the AC adapter 244 may include a transformer to convert the power received from the AC power source 242 to a lower voltage, a rectifier to convert the AC power to DC power, and/or a filter to smooth the waveform of the DC power. As illustrated, the charging station 40 may also include a cable 248 coupling the AC adapter 244 and the receiving module 120. However, in some embodiments, the charging station 40 may include a single housing (e.g., the receiving module 120 may include the AC adapter 244 and the plug 246).

As noted above, in certain embodiments, the charging station 40 may include a processor 250. The processor 250 may, for example, enable the charging station 40 to control the recharging of the power module 38, to receive the identification signal from the sensor 14 and determine whether the power module 38 is suitable to be charged by the charging station 40, and/or to cause the one or more indicator lights 152 to emit light in response to a determination of charging, fully charged, and/or failing to be charged. The charging station 40 may also include a transceiver 252 for wirelessly communicating with the sensor 14 and/or the power module 38. Additionally, in some embodiments, the charging station 40 may include a memory 254 (e.g., a tangible, non-transitory memory), which may store instructions or code for communicating with the monitor 12 and/or the sensor 14 and for recharging the power module 38. Additionally, as described above with respect to FIG. 2, the charging control circuit 110 may be configured to determine whether the power source 54 of the power module 38 is receiving charge, is fully charged, and/or is failing to be charged.

As illustrated, the receiving module 120 of the charging station 40 includes the mating connector 146, as described above with respect to FIG. 3, that is configured to couple to the connector 142 of the power module 38. However, it should be noted that the receiving module 120 may additionally or alternatively include the inductive charging port 170, and/or the inductive charging receptacle 200, as described above with respect to FIGS. 4 and 5, respectively. The charging station 40 also may include the one or more indicator lights 152 (e.g., the first and the second indicator lights 154 and 156). Additionally, in certain embodiments, the charging station 40 may include a display (not shown) and/or a speaker (not shown) to provide additional indications to a user.

As noted above, in certain embodiments, in may be desirable to recharge the power module 38 while the power module 38 is operatively coupled to and in use with the sensor 14. Accordingly, in certain embodiments, the charging station 40 may be configured to recharge the power module 38 while the power module 38 is in use with the sensor 14, as illustrated in FIG. 8. As noted above, the power module 38 may be disposed in or on the sensor body 36. However, as illustrated, the power module 38 may be external to the sensor body 36 and operatively coupled to the sensor 14 by a lead 280. This may be desirable in certain embodiments, because coupling the receiving module 120 to the power module 38 while the power module 38 is disposed in or on the sensor body 36 may cause the sensor 14 to become bulky and/or uncomfortable for the patient. That is, by separating the power module 38 from the sensor body 36, the power module 38 may be more comfortably worn by the patient, particularly during recharging.

The lead 280 may be an electrical conductor, such as a power cable, that transmits power from the power module 38 to the sensor 14. The lead 280 may terminate with the power module 38, which may be integrated into or be attached to a bracelet 282. In certain embodiments, the power module 38 may be removed from the bracelet 282. This may be desirable in some embodiments, for example, to provide a new bracelet 282 for each patient (e.g., utilize disposable bracelets 282), while enabling the power module 38 to be reused. The bracelet 282 may be, for example, a medical bracelet. Additionally, the lead 280 may be connected to and separated from the power module 38 and/or the sensor 14. That is, the lead 280 may be separable (e.g., releasable) from the power module 38, the bracelet 282, and/or the sensor 14. Alternatively, the lead 280 may be permanently affixed to the power module 38 and/or the bracelet 282. That is, in one embodiment, the power module 38 may be disposed in the bracelet 282 and coupled to the sensor 14 via the lead 280, and the power module 38 may be non-separable from the sensor 14. It should be noted, however, that the power module 38 may be disposed in any suitable object such as, for example, a garment (e.g., a shirt), a ring, a necklace, a headband, or the like.

As illustrated, the charging station 40 includes the AC power source 242, the wall-wart AC adapter 244, and the plug 246 to plug into the AC power source 242. However, it should be noted that any suitable power source 240 may be used, such as a battery and/or an energy harvesting power supply (e.g., a motion generated energy harvesting device, thermoelectric generated energy harvesting device, or the like). The charging station 40 may additionally include the cable 248 disposed between the AC adapter 244 and the receiving module 120. The receiving module 120 may be constructed to include the mating connector 146 and/or the inductive charging port 170, as described above with respect to FIGS. 3 and 4, respectively. However, in certain embodiments, it may be desirable to construct the receiving module 120 such that the receiving module 120 may be inserted into and/or disposed around the power module 38. In this manner, the receiving module 120 may be configured to attach to the power module 38 while the power module 38 is coupled to the sensor body 38 and/or the bracelet 282 (or other object). This may be desirable, for example, in embodiments in which the power module 38 is non-separable from the sensor body 36 and/or the bracelet 282. For example, as illustrated, the receiving module 120 may include a connector 284 (e.g., a power plug) that may be configured to transmit power to the power module 38. The connector 284 may be any suitable connector, such as a universal serial bus (USB) plug, a DC power plug, a coaxial DC power plug, a locking DC power plug, an AC power plug, or the like. Accordingly, the power module 38 may include a mating connector 286 configured to receive the connector 284 of the receiving module 120. Accordingly, the mating connector 286 may be a USB receptacle, a DC power receptacle, a coaxial DC power receptacle, a locking DC power receptacle, an AC power receptacle, or the like.

Because such an embodiment of the charging station 40 may be used to recharge one power module 38 at a time, rather than recharging multiple power modules 38 simultaneously, the charging station 40 may only include the first indicator light 154 to minimize cost and/or bulkiness of the charging station 40. Further, in some embodiments, the charging station 40 may not include the one or more indicator lights 152. However, it should be appreciated that the charging station 40 may include any number of indicator lights 152. As described above, the charging control circuit 110 may cause the one or more indicator lights 116 of the power module 38 to emit light in response to a determination of charging, fully charged, and/or failing to be charged. Further, the charging control circuit 110 may cause the display 118 of the power module 38 to display any suitable indication of the charging status, such as the battery indicator 134, a symbol, and/or a textual message.

In other embodiments, rather than the transmitting power directly via the connector 284, the receiving module 120 may be configured to transmit power wirelessly. For example, as illustrated in FIG. 9, the receiving module 120 of the charging station 40 includes an inductive charging clamp 290 that may be configured to be at least partially disposed around (e.g., fit about, wrap around, secure to, etc.) the power module 38 while the power module 38 is coupled to the sensor body 38 and/or the bracelet 282. Accordingly, to facilitate the coupling of the inductive charging clamp 290 and the power module 38, the power module 38 may be disposed in the bracelet 282 or sensor body 36 such that at least a portion of the housing 130 of the power module 38 extends past (e.g., protrudes from) the bracelet 282 or the sensor body 36. Additionally or alternatively, the bracelet 282 or sensor body 36 may include slots (e.g., openings) to enable the sides of the inductive charging clamp 290 to be inserted into the bracelet 282 or sensor body 36. To facilitate the power transfer, the inductive charging clamp 290 may include the primary inductor 172, and the power module 38 may include the secondary inductor 174, as described above with respect to FIG. 4.

While the embodiments described above with respect to FIGS. 8 and 9 relate to embodiments of the charging station 40 which include the cable 248 disposed between the receiving module 120 and the power source 240, the present embodiments also contemplate a cordless charging station 40. The cable 248 may act to tether the patient to the charging station 40, thus preventing unencumbered motion by the patient. Accordingly, in certain embodiments, it may be desirable to provide a cordless charging station 40 to facilitate the unencumbered motion of the patient.

For example, FIG. 10 illustrates a cordless embodiment of the charging station 40 that may be configured to be affixed to the patient (e.g., a patient-wearable charging station 40). In particular, to facilitate the cordless operation of the charging station 40 (e.g., to operate without the cable 248 disposed between the receiving module 120 and the power source 240), the charging station 40 may include a housing 300 configured to house the components of the charging station 40, such as the power transmission module 106, the receiving module 120, the processor 250, the transceiver 252, the memory 254, and the power source 240. Accordingly, the power source 240 may be a cordless power source, such as battery (e.g., a rechargeable battery) and/or an energy harvesting power supply (e.g., a motion generated energy harvesting device or thermoelectric generated energy harvesting device). In this manner, the cable 248 may not be needed to transmit power from the power source 240 to other components of the charging station 40.

Additionally, to facilitate the cordless operation, the housing 300 that may be configured to wrap around a portion of the patient (e.g., the wrist or the finger) and/or at least a portion of the bracelet 282 to position the receiving module 120 near the power module 38. That is, the housing 300 may position the receiving module 120 at a distance from the power module 38, when the housing 300 is coupled to the patient or the bracelet 282, such that the receiving module 120 may couple to the power module 38 without providing a cable between the housing 300 and the receiving module 120 to extend the “reach” of the receiving module 120. It should be noted that in other embodiments, the charging station 40 may additionally or alternatively be configured to wrap around the sensor body 36 and/or any other object (e.g., a garment, a ring, a necklace, a headband) that may be utilized instead of, or in addition to, the bracelet 282. In certain embodiments, no cables may be present between the housing 300 and the receiving module 120. Accordingly, the housing 300 may be at least partially flexible and/or elastic. The housing 300 may be constructed from any suitable materials, such as fabric, rubber, or elastomeric compositions (including acrylic elastomers, polyimide, silicones, silicone rubber, celluloid, PMDS elastomer, polyurethane, polypropylene, acrylics, nitrile, PVC films, acetates, and latex).

To secure the charging station 40 about the patient or the bracelet 282, the housing 300 may also include one or more fasteners 302, such as buttons, snap fasteners, hook and loop fasteners, clips, zippers, magnets, or the like. In certain embodiments, the housing 300 may be configured to wrap completely around a portion of the patient, and the one or more fasteners 302 may be configured to affix to each other. In other embodiments, the housing 300 may be configured to be disposed about a portion of the bracelet 282. In such embodiments, the one or more fasteners 302 of the charging station 40 may be configured to couple to corresponding (e.g., matching) fasteners 304 disposed on the bracelet 282. Accordingly, the fasteners 304 may be buttons, snap fasteners, hook and loop fasteners, clips, zippers, magnets, or the like.

In certain embodiments, the receiving module 120 may be constructed to include the mating connector 146 and/or the inductive charging port 170, as described above with respect to FIGS. 3 and 4, respectively. As illustrated, the receiving module 120 may include the connector 284 (e.g., a power plug) and/or the inductive charging clamp 290, as described above with respect to FIGS. 8 and 9, respectively. As illustrated, in certain embodiments, the receiving module 120 may include the connector 284 and may be a USB plug 306. Accordingly, in these embodiments, the power module 38 may include the mating connector 286, which may be a USB receptacle 308.

Additionally, similar to FIGS. 8 and 9, the embodiment of the charging station 40 as illustrated in FIG. 10 may be used to recharge one power module 38 at a time, rather than recharging multiple power modules 38 simultaneously. As such, the charging station 40 may only include the first indicator light 154 to minimize cost and/or bulkiness of the charging station 40. Further, in some embodiments, the charging station 40 may not include the one or more indicator lights 152. However, it should be appreciated that the charging station 40 may include any number of indicator lights 152. Additionally, as described above, the charging control circuit 110 may cause the one or more indicator lights 116 of the power module 38 to emit light in response to a determination of charging, fully charged, and/or failing to be charged. Further, the charging control circuit 110 may cause the display 118 of the power module 38 to display any suitable indication of the charging status, such as the battery indicator 134, a symbol, and/or a textual message. As such, in certain embodiments, it may be desirable to position the power module 38 in and/or on the bracelet 282, if utilized, such that the one or more indicator lights 116 and/or the display 118 may be easily viewed and not covered by a portion of the bracelet 282.

The disclosed embodiments may be interfaced to and controlled by a computer readable storage medium having stored thereon a computer program. The computer readable storage medium may include a plurality of components such as one or more of electronic components, hardware components, and/or computer software components. These components may include one or more computer readable storage media that generally store instructions such as software, firmware and/or assembly language for performing one or more portions of one or more implementations or embodiments of an algorithm as discussed herein. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or solid-state or electronic memory. Other forms of non-transitory and/or tangible computer readable storage media not list may be employed with the disclosed embodiments.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A system for measuring a physiological condition of a patient, comprising:
a wireless sensor configured to generate a physiological signal of a patient, wherein the wireless sensor comprises a power module comprising a power source configured to power the wireless sensor and comprising a charging control circuit configured to compare a level of charge of the power source to a threshold and to provide a low power indication in response to determining that the level of charge is below the threshold; and
a charging device comprising a receiving module configured to couple to the power module and comprising a power transmission module configured to recharge the power source when the power module is coupled to the receiving module.

2. The system of claim 1, comprising a monitor configured to receive the physiological signal from the wireless sensor, wherein the monitor comprises a processor configured to calculate a physiological parameter of the patient based at least in part upon the received physiological signal, and wherein the monitor comprises the charging device.

3. The system of claim 2, wherein the wireless sensor comprises a pulse oximetry sensor, and wherein the physiological parameter comprises blood oxygen saturation.

4. The system of claim 2, wherein the processor is configured to monitor the level of charge of the power source and to provide an indication of completion of charge in response to a determination that the power source is fully charged.

5. The system of claim 4, wherein the monitor comprises an indicator light, and wherein the processor is configured to cause the indicator light to emit light in response to the determination that the power source is fully charged.

6. The system of claim 2, comprising a dongle configured to plug into a sensor port of the monitor, and wherein the dongle comprises the receiving module.

7. The system of claim 6, wherein the dongle comprises a transceiver configured to wirelessly receive the physiological signal from the wireless sensor and to transmit the physiological signal to the processor of the monitor.

8. The system of claim 7, wherein the dongle is configured to receive emitter driving signals from the monitor and to wirelessly transmit the emitter driving signals to the wireless sensor.

9. The system of claim 1, wherein the power module is removable from the wireless sensor and comprises a plug, and wherein the receiving module comprises a receptacle configured to receive the plug of the power module.

10. The system of claim 1, wherein the power transmission module comprises a first inductor, and wherein the power module comprises a second inductor configured to receive electromagnetic charging signals from the first inductor.

11. The system of claim 10, wherein the receiving module comprises a clamp configured to be at least partially disposed about the power module, a port configured to receive the power module via a snap-in connection, or a receptacle configured to house the power module.

12. The system of claim 1, wherein the receiving module is configured to couple to the power module while the power module is operatively coupled to the wireless sensor.

13. The system of claim 12, wherein the charging device comprises a patient-wearable charging device.

14. The system of claim 12, wherein the power module is non-separable from the wireless sensor.

15. The system of claim 12, comprising:
a bracelet housing the power module, wherein the bracelet is configured to be removably secured to the patient; and
a lead coupling the power module to the wireless sensor; and
wherein the charging device is cordless and is configured to be removably attached to the bracelet.

16. A wireless sensor, comprising:
one or more sensing components configured to generate a physiological signal of a patient;
a transceiver configured to transmit the physiological signal;
a power source configured to power the one or more sensing components and the transceiver and to be recharged based at least in part upon power received from a charging device; and
a charging control circuit configured to monitor a level of charge of the power source and to provide a first indication in response to determining that the level of charge is below a predetermined threshold and a second indication in response to determining that the power source is fully charged.

17. The wireless sensor of claim 16, comprising a first housing configured to house the one or more sensing components and comprising a second housing configured to house the transceiver, the power source, and the charging control circuit, and wherein the second housing is removable from the first housing.

18. The wireless sensor of claim 17, wherein the first housing is disposable.

19. The wireless sensor of claim 17, wherein the second housing is external to the first housing and is coupled to the first housing via a lead.

20. The wireless sensor of claim 16, comprising a sensor body configured to house the one or more sensing components, the transceiver, the power source, and the charging control circuit, and wherein the one or more sensing components, the transceiver, the power source, and the charging control circuit are non-removable from the sensor body.

21. The wireless sensor of claim 16, comprising a display, wherein the charging control circuit is configured to cause the display to display the first indication or the second indication.

22. The wireless sensor of claim 21, wherein the display comprises a battery indicator configured to provide an indication of the level of charge of the power source.

23. The wireless sensor of claim 21, wherein the display comprises an electronic ink (E-ink) display.

24. A charging device, comprising:
a power source;
a first receiving module configured to couple to a first power module of a medical sensor;
a power transmission module configured to receive power from the power source and to transmit power to the first power module when the first power module is coupled to the first receiving module; and
processing circuitry configured to monitor a level of charge in the first power module and to cause a first indicator light to emit light in response to a determination that the first power module is fully charged.

25. The charging device of claim 24, comprising a second receiving module configured to couple to a second power module of a medical sensor, wherein the power transmission module is configured to transmit power to the second receiving module when the second power module is coupled to the second receiving module, and wherein the processing circuitry is configured to monitor a level of charge of the second power module and to cause a second indicator light to emit light in response to a determination that the second power module is fully charged.

26. The charging device of claim 24, wherein the charging device comprises a power adapter or a pulse oximetry monitor.

27. A method of retrofitting a patient monitor, comprising:
providing a dongle comprising a receiving module configured to couple to a power module of a sensor; and
coupling the dongle to a port of a patient monitor, wherein the dongle is configured to receive power from the patient monitor when the dongle is coupled to the patient monitor and to provide the received power to the power module of the sensor for recharging the power module when the sensor is coupled to the receiving module of the dongle.

28. The method of claim 27, wherein the dongle comprises a wireless transceiver, and wherein the dongle is configured to wirelessly receive signals from the sensor and to transmit the received signals to the patient monitor when the dongle is coupled to the patient monitor.
